(19) United States
(12) Patent Application Publication
(10) Pub. No.: US 2024/0026277 A1
(43) Pub. Date: Jan. 25, 2024

(54) CELL CULTURE CHAMBER FOR INCREASED CELL YIELD

(71) Applicant: OCTANE BIOTECH INC., Kingston (CA)

(72) Inventors: Nuala TRAINOR, Kingston (CA); Michael NUTTALL, Kingston (CA); Peter SZARAZ, Kingston (CA); Carly PETES, Kingston (CA); Matteo DA ROS, Kingston (CA); Ashkan TABIBIAN, Kingston (CA); James MARSHALL, Kingston (CA); Cohen O'GRADY, Kingston (CA)

(21) Appl. No.: 18/354,770

(22) Filed: Jul. 19, 2023

Related U.S. Application Data
(60) Provisional application No. 63/495,873, filed on Apr. 13, 2023.
(60) Provisional application No. 63/368,942, filed on Jul. 20, 2022.

(51) Int. Cl. C12M 1/36; C12M 1/04; C12M 1/00

(52) U.S. Cl. CPC C12M 41/48; C12M 23/24; C12M 23/02

(57) ABSTRACT

The present disclosure provides cell culture chambers for use in automated cell engineering systems, and in particular, cell culture chambers that provide increased cell yield. A cell culture chamber may include a first body forming a first portion of the cell culture chamber. A cell culture chamber may include a second body forming a second portion of the cell culture chamber, the first body and the second body configured to couple together, forming an enclosed volume, wherein a first non-porous, gas-permeable material is disposed on the first body; and wherein the first non-porous, gas-permeable material and the first body are formed together. A second non-porous, gas-permeable material may be disposed on the second body, and the second non-porous, gas-permeable material and the second body may be formed together. The cell culture chamber may further include at least one bubble trap integrally formed with the first body and/or the second body.

### FIELD OF THE INVENTION

The present disclosure provides cell culture chambers for use in automated cell engineering systems, and in particular, cell culture chambers that provide increased cell yield.

### BACKGROUND OF THE INVENTION

As anticipation builds about accelerated clinical adoption of advanced cell therapies, more attention is turning to the underlying manufacturing strategies that will allow these therapies to benefit patients worldwide. While cell therapies hold great promise clinically, high manufacturing costs relative to reimbursement present a formidable roadblock to commercialization. Thus, the need for cost effectiveness, process efficiency and product consistency is driving efforts for automation in numerous cell therapy fields, and particularly for T cell immunotherapies.

Integration of cell activation, transduction and expansion into a commercial manufacturing platform is critical for the translation of these important immunotherapies to the broad patient population. For these life-saving treatments to be applicable to the global patient population, a shift in manufacturing techniques must be implemented to support personalized medicine. The benefits of automation include labor time savings associated with using automation as well as improved product consistency, decreased room classification, decreased clean room footprint, decreased training complexities, and improved scale-up and tracking logistics. Furthermore, software can be used to streamline the documentation processes by using automatically generated electronic batch records to provide a history of all processing equipment, reagents, patient identification, operator identification, in-process sensor data, etc.

What is needed to advance these therapies and automated systems are components of cell expansion systems, such as cell culture chambers, that increase cell output, or provide desired cellular characteristics. The present application fulfills these needs.

### SUMMARY OF THE INVENTION

In some aspects, the techniques described herein relate to a cell culture chamber for use in an automated cell engineering system, the cell culture chamber including: a first body forming a first portion of the cell culture chamber; a second body forming a second portion of the cell culture chamber, the first body and the second body configured to couple together, thereby forming an enclosed volume; wherein a first non-porous, gas-permeable material is disposed on the first body; and wherein the first non-porous, gas-permeable material and the first body are formed together.

In some aspects, the techniques described herein relate to a cell culture chamber, further including a second non-porous, gas-permeable material disposed on the second body; and wherein the second non-porous, gas-permeable material and the second body are formed together.

In some aspects, the techniques described herein relate to a cell culture chamber, further including at least one bubble trap integrally formed with the cell culture chamber.

In some aspects, the techniques described herein relate to a cell culture chamber, further including one or more structural islands.

In some aspects, the techniques described herein relate to a cell culture chamber, wherein the first body and the second body couple together using mating elements.

In some aspects, the techniques described herein relate to a cell culture chamber, wherein the first body includes a plurality of openings configured to allow gas exposure to an external environment via an outside surface of the first non-porous, gas-permeable material and/or the second non-porous, gas-permeable material.

In some aspects, the techniques described herein relate to a cell culture chamber, wherein the plurality of openings include a geometric, hexagonal, septagonal, octagonal, symmetrical, honeycomb, round, circular, oblong, oval, or square shape.

In some aspects, the techniques described herein relate to a cell culture chamber, wherein the first non-porous, gas-permeable material is positioned on an inner surface of the first body, the first non-porous, gas-permeable material being configured to cover the plurality of openings of the first body.

In some aspects, the techniques described herein relate to a cell culture chamber, wherein a structural section of the second body includes a plurality of openings configured to allow gas exposure to an external environment via an outside surface of the first non-porous, gas-permeable material and/or the second non-porous, gas-permeable material.

In some aspects, the techniques described herein relate to a cell culture chamber, wherein the plurality of openings include a geometric, hexagonal, septagonal, octagonal, symmetrical, honeycomb, round, circular, oblong, oval, or square shape.

In some aspects, the techniques described herein relate to a cell culture chamber, wherein the second non-porous, gas-permeable material is positioned on an inner surface of the first body, the second non-porous, gas-permeable material being configured to cover the plurality of openings of the second body.

In some aspects, the techniques described herein relate to a cell culture chamber, wherein the at least one bubble trap is integrally formed with the first body of the cell culture chamber, and further including a bubble trap track integrally formed with the first body and fluidly connected to the at least one bubble trap.

In some aspects, the techniques described herein relate to a cell culture chamber, wherein at least two bubble traps are integrally formed with the first body of the cell culture chamber, and the bubble trap tracks are fluidly connected to the at least two bubble traps.

In some aspects, the techniques described herein relate to a cell culture chamber, wherein the bubble trap track tapers in width.

In some aspects, the techniques described herein relate to a cell culture chamber, wherein the bubble trap track tapers in height.

In some aspects, the techniques described herein relate to a cell culture chamber, wherein at least two bubble traps are integrally formed with the first body of the cell culture chamber, and at least two corresponding bubble trap tracks are fluidly connected to each of the at least two bubble traps such that the at least two corresponding bubble trap tracks are disconnected from each other.

In some aspects, the techniques described herein relate to an automated cell engineering system, including: a cassette including: a cell culture chamber having at least one bubble trap disposed thereon, and further including: a first body forming a first portion of the cell culture chamber; a second body forming a second portion of the cell culture chamber, the first body and the second body configured to couple together, thereby forming an enclosed volume; wherein a first non-porous, gas-permeable material is disposed on the first body and/or a second non-porous, gas-permeable material is disposed on the second body; and wherein the first non-porous, gas-permeable material and the first body are formed together and the second non-porous, gas-permeable material and the second body are formed together.

In some aspects, the techniques described herein relate to an automated cell engineering system, the cell culture chamber is in a horizontal orientation.

In some aspects, the techniques described herein relate to an automated cell engineering system, wherein the cell culture chamber includes a plurality of bubble traps disposed thereon.

In some aspects, the techniques described herein relate to an automated cell engineering system, wherein the first body includes a plurality of openings, and the second body includes a plurality of openings.

In some aspects, the techniques described herein relate to an automated cell engineering system, wherein the first non-porous, gas-permeable material is positioned on an inner surface of the first body, the first non-porous, gas-permeable material being configured to cover the plurality of openings of the first body.

In some aspects, the techniques described herein relate to an automated cell engineering system, wherein the second non-porous, gas-permeable material is positioned on an inner surface of the second body, the second non-porous, gas-permeable material being configured to cover the plurality of openings of the second body.

In some aspects, the techniques described herein relate to an automated cell engineering system including: a cell culture chamber including: a first body forming a first portion of the cell culture chamber and having a plurality of bubble traps disposed thereon, and a bubble trap track disposed between and connected to the plurality of bubble traps; a second body forming a second portion of the cell culture chamber, the first body and the second body configured to couple together, thereby forming an enclosed volume; wherein a first non-porous, gas-permeable material is disposed on the first body and a second non-porous, gas-permeable material is disposed on the second body; and wherein the first non-porous, gas-permeable material and the first body are formed together and the second non-porous, gas-permeable material and the second body are formed together.

In some aspects, the techniques described herein relate to an automated cell engineering system, wherein the cell culture chamber is in a horizontal orientation.

In some aspects, the techniques described herein relate to an automated cell engineering system, wherein the bubble trap track tapers in height and/or tapers in width.

In some aspects, the techniques described herein relate to an automated cell engineering system, wherein each of the plurality of bubble traps are formed at opposite ends of the first body.

In some aspects, the techniques described herein relate to an automated cell engineering system, wherein the first non-porous, gas-permeable material is positioned on an inner surface of the first body, the first non-porous, gas-permeable material being configured to cover a plurality of openings of the first body.

In some aspects, the techniques described herein relate to an automated cell engineering system, wherein the second non-porous, gas-permeable material is positioned on an inner surface of the second body, the second non-porous, gas-permeable material being configured to cover the plurality of openings of the second body.

In some aspects, the techniques described herein relate to a cell culture chamber for use in an automated cell engineering system, the cell culture chamber including: at least one body forming an enclosed volume; and a non-porous, gas-permeable material disposed on the at least one body such that the non-porous, gas-permeable material and the at least one body are formed together; wherein no structures are disposed within the enclosed volume.

### BRIEF DESCRIPTION OF THE FIGURES

FIG. 1 shows a cassette for use in an automated cell engineering system as described herein.

FIG. 2 shows a first view of a cell culture chamber as described herein.

FIG. 3 shows a second view of a cell culture chamber as described herein.

FIG. 4 shows an internal view of a cell culture chamber as described herein.

FIG. 5 shows components of an automated cell engineering system as described herein.

FIG. 6 shows a satellite chamber of an automated cell engineering system as described herein.

FIG. 7 shows a cross-sectional view of another embodiment of a cell culture chamber for use in an automated cell engineering system as described herein.

FIG. 8 shows a second cross-sectional view of a cell culture chamber as described herein.

FIG. 9 shows a third view of a cell culture chamber as described herein.

FIG. 10 shows a fourth view of a cell culture chamber as described herein.

FIG. 11 shows a fifth view of a cell culture chamber as described herein.

FIG. 12 shows a sixth view of a cell culture chamber as described herein.

FIGS. 13A-13C depict various embodiments of at least one bubble trap and bubble trap track integrally formed with the cell culture chamber of FIG. 7.

### DETAILED DESCRIPTION OF THE INVENTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the context clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

An automated cell engineering system 102 as described herein is a fully enclosed, automated system, for performing activating, transducing, expanding, concentrating, and/or harvesting steps, of cell cultures. Automated cell engineering systems (also called automated biologic processing units throughout) provide for the automated production of cell cultures. As used herein “cell cultures” refers to any suitable cell type, including individual cells, as well as multiple cells or cells that may form into tissue structures. Exemplary cell cultures include blood cells, skin cells, muscle cells, bone cells, cells from various tissues and organs, etc. In embodiments, genetically modified immune cells, including CAR T cells, as described herein, can be produced. Exemplary automated cell engineering systems are also called COCOON®, or COCOON® system throughout (see e.g., U.S. Published Patent Application No. 2019/0169572, the disclosure of which is incorporated by reference herein in its entirety).

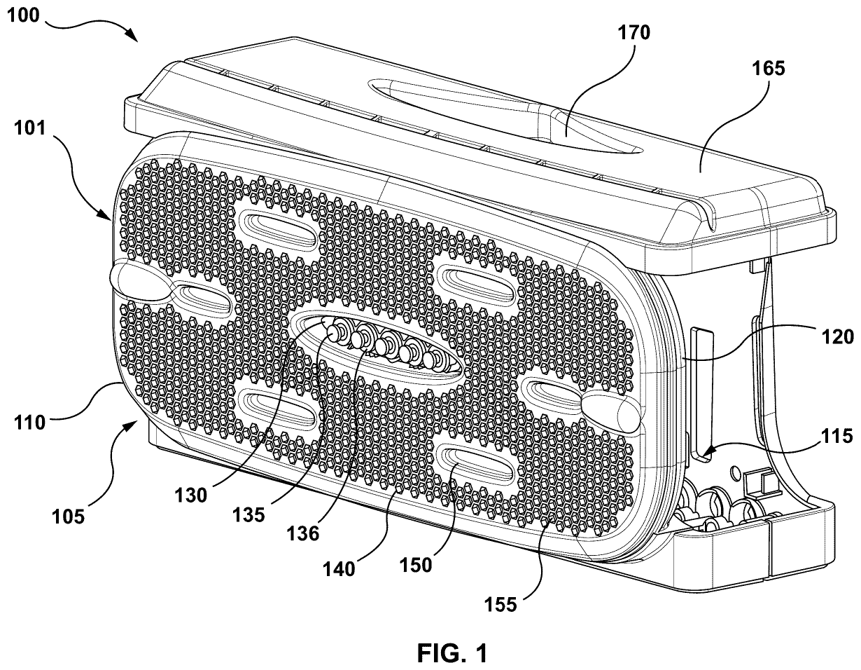

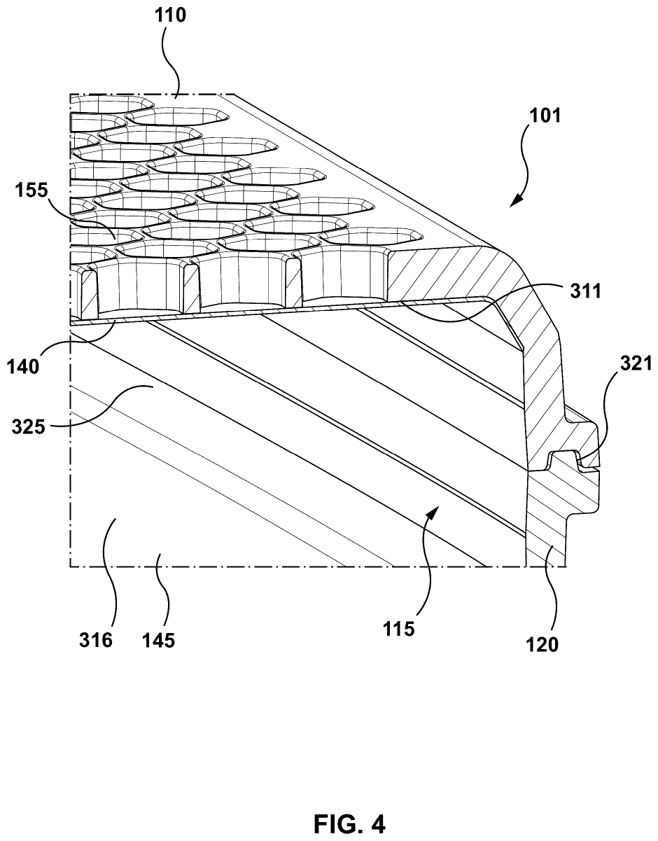

A cell culture chamber 101 for use in an automated cell engineering system includes a first body 110 and a second body 120 configured to couple together, for example, as shown in FIG. 1 and FIG. 4. The first body 110 forms a first portion 105 of the cell culture chamber 101 and the second body 120 forms a second portion 115 of the cell culture chamber 101. When coupled together, the first body 110 and the second body 120 form an enclosed volume 325. At least one structural member 130 extends from the first body 110, through the enclosed volume 325, to the second body 120, see FIG. 1 and FIGS. 2-3. The structural member 130 includes a series of lumina 135 that span from the first body 110 to the second body 120, through the enclosed volume 325. A first non-porous, gas-permeable material 140 is disposed on the first body 110 and a second non-porous, gas-permeable material 145 is disposed on the second body 120, see FIG. 4. In an embodiment, the first non-porous, gas-permeable material 140 may be formed together with the first body 110, and the second non-porous, gas-permeable material 145 may be formed together with the second body 120, the forming procedure which is described in greater detail below.

In a further embodiment, the cell culture chamber includes at least one body 110′ forming an enclosed volume 325′. The at least one body may be formed as a single, solid element that creates the enclosed volume 325′, the enclosed volume which further contains a gas-permeable material 140′ disposed thereon. At least one structural member 130′ extends from the at least one body 110′ through the enclosed volume 325′. A series of lumina 135′ further span within the enclosed volume of the at least one body. As described herein, the enclosed volume 325′ and the gas permeable material 140′ are suitably prepared using a dual-shot molding technique.

As noted above, the enclosed volume 325 is formed in the area between the first body 110 and the second body 120. The enclosed volume 325 is configured to contain a cell culture and is configured to be fluidly sealed (does not allow liquid to flow into or out of the enclosed volume, but will allow gas flow, as described herein). As shown in FIG. 4, the first body 110 and the second body 120 are coupled together using mating elements, for example at coupling 321, such as snap-fit, form-fit, or other methods known in the art. Additionally, it is envisioned, that the first body 110 and the second body 120 may be formed as a unitary body, removing the need for the first body 110 and the second body 120 to be coupled to together.

The structural member 130 extends from the first body 110, through the enclosed volume 325, to the second body 120, and includes a first side 132 and a second side 134. The first side 132 is positioned at and formed as part of the first body 110 and the second side 134 is positioned at and formed as part of the second body 120. The structural member 130 is configured to maintain integrity of the enclosed volume 325 while also defining the lumina 135 through the enclosed volume 325. In some embodiments, lumina 135 are configured to receive a valve 136 or a coupler that may be configured to be connected to an automated cell engineering system. Such components allows for external machinery, such as pumps, tubing, or other machinery, to interact with and provide additional to support to the media and cells within the enclosed volume 325 as well as the components of the automated cell engineering system. It is envisioned, the series of lumina 135 are configured to receive said valves or couplers. Valves 136 can be, for example, trumpet valves—i.e., valves that have two positions (open “0” or closed “1”) and are capable of compressing or pinching flow-paths. Lumina can also include couplers (not shown) that allow for the connection of various syringes, sampling devices, etc.

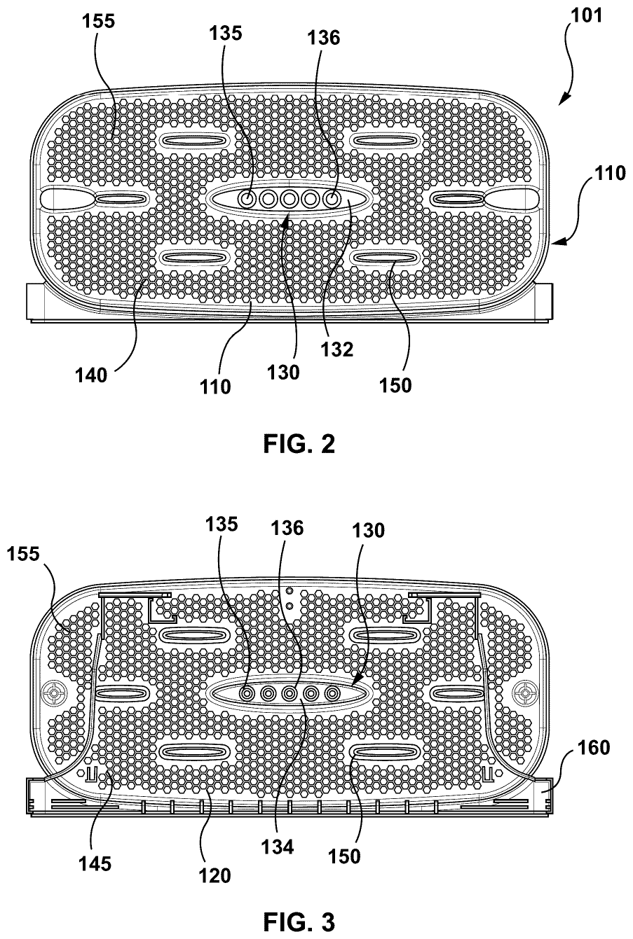

As shown in FIGS. 2 and 3, in some embodiments, the cell culture chamber 101 includes at least one or more structural islands 150 which extend from the first body 110, into the enclosed volume 325, to the second body 120. In an embodiment, each of the first body 110 and second body 120 can both include structural islands 150 that extend into the enclosed volume 325. Each structural island 150 provides additional structural support to the cell culture chamber 101 and allows for a greater volume to be positioned within the cell culture chamber 101 without comprising the structural integrity. In addition, the structural islands 150 provide a greater surface area within the cell culture chamber for the cells to rest upon, allowing for an increase in growth. Structural islands 150 can also provide increased turbulent fluid flow (i.e. non-laminar) during filling and movement of fluid within the cell culture chamber to assist with cell mixing and/or removal from adherent surfaces. In embodiments, a lumen may be positioned within the structural islands 150 as to allow for air to flow around and through the cell culture chamber and the cassette, providing an increase in potential cooling and temperature management of the cassette.

In order to allow gas exchange between the enclosed volume 325 and the outside environment, the first body 110 and/or the second body 120 may include a plurality of openings 155 configured to allow for gas exposure to the external environment via the outside surface of the first non-porous, gas-permeable material 140 and/or the second non-porous, gas-permeable material 145 disposed in the cell culture chamber 101. The plurality of openings 155 may take a variety of shapes including geometric, symmetrical, honeycomb, round, circular, oblong, oval, square, etc. Additionally, the design of the plurality of openings 155 may be used to provide additional structural support and strength to the first body 110 and the second body 120. For example, the use of a honeycomb structure allows for a high ratio of open space to structure, allowing for a higher volume of gas to be exchanged, while reducing the amount of structure that is required to support the first body 110 and/or the second body 120.

An inner surface 311, otherwise known as a media-contacting surface, of the first body 110 and an inner surface 316, or media-contacting surface, of the second body 120 of the cell culture chamber 101 are suitably lined or covered with non-porous gas-permeable materials so as to prevent water or other liquids from evaporating from within the cell culture chamber 101. As used herein, “a non-porous, gas-permeable material” means any composition film or material used for gas-permeable cell culture devices, that allows for gases to pass and enter the cell culture chamber 101, but does not contain pores or holes that allow for passage or leakage of liquids (i.e. cell media). Exemplary non-porous, gas-permeable materials include, but are not limited to, silicone, fluoroethylene polypropylene (FEP), polyolefin, ethyl vinyl olefin (EVO) and ethylene vinyl acetate copolymer. Non-porous, gas-permeable materials as described herein suitably help to deliver one or more gasses, including oxygen, nitrogen, CO₂, etc., to the cells in the cell culture chamber 101. Additionally, the width of the non-porous gas-permeable material may be reduced or increased in order to increase or decrease the gas exchange through the material.

As shown in FIG. 4, in embodiments, a first non-porous, gas permeable layer or material 140 is positioned at the inner surface 311 of the first body 110 and a second non-porous, gas permeable material 145 is positioned at the inner surface 316 of the second body 120. During the formation of the cell culture chamber 101, for example, through the use of molding, the first and second non-porous gas-permeable layers 140/145 may be simultaneously created at the inner surfaces 311/316 through a dual-shot molding technique. In an embodiment, this molding technique includes an injection molding technique. For example, the first body 110 and second body 120 of the cell culture chamber 101 are formed via a first shot of the dual-shot molding technique. After a brief delay allowing the thermoplastic to harden, a core portion of the mold is retracted in small volumes to create space for a second shot of silicone (or any other suitable gas-permeable material) to form the gas permeable material 140/145 within the first body 110 and second body 120, respectively. The measured thickness of the gas permeable material 140/145 is determinative on the amount of mold retracted from the first shot, and thus can be adjusted in accordance with end user needs. Additionally, the non-porous, gas-permeable materials may be cast onto or into the cell culture chamber 101, to provide the gas-permeable characteristics described herein. Casting of the non-porous, gas-permeable material may be carried out using various methods known in the art. Through the process of casting the non-porous, gas-permeable material onto or into the cell culture chamber 101, the structural strength and integrity of the gas-permeable membrane may be increased, while maintaining gas-permeable characteristics. The non-porous, gas-permeable materials may also be coated, sprayed, painted, layered, or otherwise disposed on the inner surfaces 311/316 of the first 110 and second 120 bodies. The first and second non-porous, gas-permeable layers 140/145 may be constructed of the same or different materials, and, in embodiments, each layer 140/145 may include a plurality of different materials.

In exemplary embodiments, “a portion” of the inner surface, such as 311, comprises the non-porous, gas-permeable material. As used herein, “a portion” refers to at least about 20% of the surface, being made up of the non-porous, gas-permeable material. In embodiments, both media contacting surfaces 311 and 316 comprise the non-porous, gas-permeable material. In such embodiments in which less than 100% of the media-contacting surface is made of the non-porous, gas-permeable material, the remainder of the media-contacting surface 311 can include other suitable materials, including various plastics that promote the adhesion and growth of cells (e.g., polypropylene, polystyrol, polystyrene, etc.). In embodiments in which less than 100% of the media-contacting surface is made of the non-porous, gas-permeable material, the remainder of the media-contacting surface 316 can include other suitable materials, including various plastics that provide structural support (e.g., polypropylene, polystyrol, polystyrene, etc.). In an embodiment, the media contacting surface or inner surface 311 of the first body 110 may comprise a thermoplastic suitable for cell culture, in which case the first body 110 provides structure and encloses the chamber volume without gas permeable material. The first body 110 of the cell culture chamber 101 may suitably be made of a transparent material that allows for visual inspection or imaging of the cell culture within the cell culture chamber 101.

In embodiments, at least about 30% of the media-contacting surface is made up of the non-porous, gas-permeable material, more suitably at least about 40%, at least about 50%, at least about 60%, at least about 70%, at least about 80%, at least about 90%, or at least about 100% (i.e., the entirety) of the media-contacting surface comprises the non-porous, gas-permeable material.

In embodiments, the inner surfaces of the first body 110 and the second body 120 may further include a surface coating such as a surface coating that activates a cell; a surface coating that modulates a biological pathway in a cell; a surface coating that enhances growth of a cell; a surface coating that increases transduction efficiency of a cell; a surface coating that improves selection for a certain type of cells; a surface coating that improves adhesion of a cell; a surface coating that inhibits a cell; a surface coating that has controlled solubility. Thus, in embodiments, the non-porous gas-permeable material that suitably makes up a portion or the entirety of the media-contacting surface can further include a surface coating as described herein. In other embodiments, the remainder of the media-contacting surface that does not include a non-porous gas-permeable material can also include a surface coating as described herein. In other embodiments, the entire media-contacting surface does not include a non-porous gas-permeable material, but still includes a surface coating as described herein. The adhesion of a cell can also provide an opportunity for cell selection to promote the growth of a desired population, while not allowing adherence of an undesired population.

As used herein, a “surface coating that activates a cell” refers to a material, substrate or component that causes a cell to proliferate and/or differentiate.

As used herein, a “a surface coating that modulates a biological pathway in a cell” refers to a material, substrate or component that causes one or more actions among molecules in a cell, resulting in a certain product or change in the cell. For example, such surface coatings can trigger the assembly of new molecules, such as a fat or protein, turn genes on and off, or cause a cell to move.

As used herein, “a surface coating that enhances growth of a cell” refers to a material, substrate or component that causes the cell to grow faster or in a greater number, than in the absence of the material.

As used herein, “a surface coating that improves adhesion of a cell” refers to a material, substrate or component that causes the cell to better interact with and attach to a surface, and also interact with other cells, as they adhere to a surface.

As used herein, “a surface coating that inhibits a cell” refers to a material, substrate or component that causes the cell to not grow and/or not adhere to a media-contacting surface.

As used herein “a surface coating that responds to media conditions” refers to a material, substrate or component that changes when a media condition changes. Exemplary changes include changes in temperature, pH, oxygen level or concentration, level of toxic gases, presence of toxic substances, and include for example a color change as a monitoring aid.

As used herein “a surface coating that has controlled solubility” refers to a material, substrate or component that releases from the surface at a particular time or in response to a particular temperature or pH, for example, to attain controlled release of coating contents.

A surface treatment (e.g. corona, plasma, etching, etc.) can be used to facilitate the binding (or rejection) of a coating. This can allow for selective surface modifications to enable two different types of cells to be grown in the same cell culture chamber, or for selective unit operations within the same chamber.

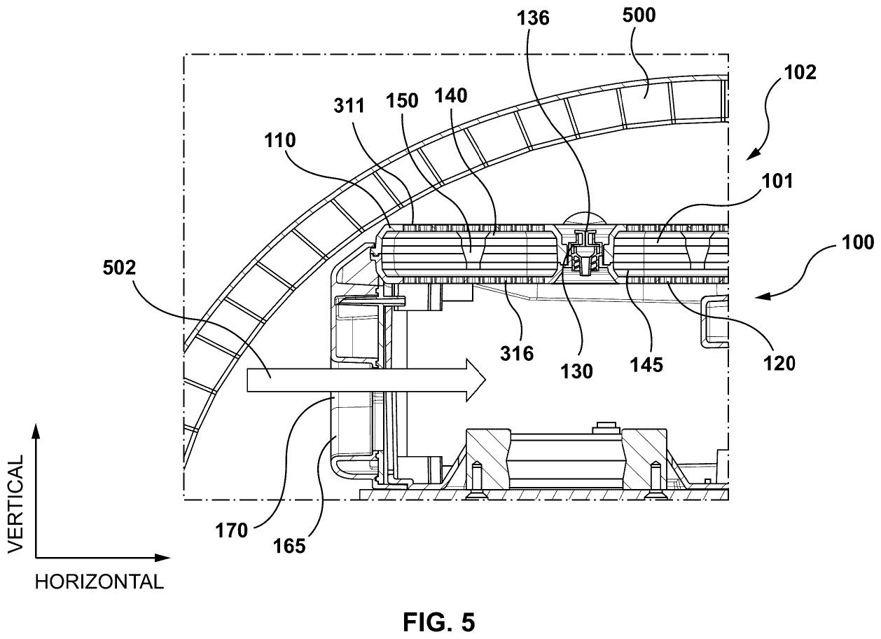

As shown in FIG. 1, in some embodiments the cell culture chamber 101 is configured to be positioned as part of a cassette 100 for use in an automated cell engineering system 102 (see FIG. 5). As used herein a “cassette” refers to a largely self-contained, removable and replaceable element of a cell engineering system 102 that includes one or more chambers for carrying out the various elements of cell production, and suitably also includes one or more of a cell media, an activation reagent, a vector, etc.

Cassette 100 includes a low temperature chamber, suitable for storage of a cell culture media, as well as a high temperature chamber, suitable for carrying out activation, transduction and/or expansion of a cell culture, including an immune cell culture. High temperature chamber may be separated from low temperature chamber by a thermal barrier. As used herein “low temperature chamber” refers to a chamber, suitably maintained below room temperature, and more suitably from about 4° C. to about 8° C., for maintenance of cell media, etc., at a refrigerated temperature. The low temperature chamber can include a bag or other holder for media, including about 1 L, about 2 L, about 3 L, about 4 L, or about 5 L of fluid. Additional media bags or other fluid sources can be connected externally to the cassette and connected to the cassette via an access port. The cassette further includes one or more fluidics pathways connected to the cell culture chamber, wherein the fluidics pathways provide recirculation, removal of waste and homogenous gas exchange and distribution of nutrients to the cell culture chamber without disturbing cells within the cell culture chamber. Cassette 100 also further includes a pumping system comprising one or more pumps, including peristaltic pumps, for driving fluid through the cassette, as described herein, as well as one or more valves, for controlling the flow through the various fluidic pathways.

In embodiments, the cell culture chamber 101 is a contained and non-flexible container. The use of a non-flexible chamber allows the cells to be maintained in a substantially undisturbed state. As described, cell culture chamber 101 can be oriented so as to allow the immune cell culture to spread throughout the cell culture chamber. As shown in FIG. 5, cell culture chamber 101 is suitably maintained in a horizontal position that is parallel with the floor or table, maintaining the cell culture in an undisturbed state, allowing the cell culture to spread across a large area of the cell culture chamber. In embodiments, the overall thickness of cell culture chamber 101 is on the order of about 0.5 cm to about 5 cm. Suitably, the cell culture chamber may have a volume of between about 0.50 ml and about 1 L, more suitably about 0.5 to about 900 ml, or the cell culture chamber has a volume of about 825 ml. Suitably, the cell culture chamber may have a volume of between about 0.5 ml to about 500 ml, about 10 ml to about 300 ml, more suitably between about 50 ml and about 200 ml, or the cell culture chamber has a volume of about 180 ml. The use of a low chamber height (less than 5 cm, suitably less than 4 cm, less than 3 cm, or less than 2 cm) allows for effective media and gas exchange in close proximity to the cells. Ports are configured to allow mixing via recirculation of the fluid without disturbing the cells. Larger height static vessels can produce concentration gradients, causing the area near the cells to be limited in oxygen and fresh nutrients. Through controlled flow dynamics, media exchanges can be performed without cell disturbance. Media can be removed from the additional chambers (no cells present) without risk of cell loss.

As described herein, in exemplary embodiments the cassette is pre-filled with one or more of a cell culture, a culture media, an activation reagent, and/or a vector, including any combination of these. In further embodiments, these various elements can be added later via suitable injection ports, etc.

The cell engineering system 102 may include components such as a gas control seal, warming zone, actuators, pivot for rocking or tilting the cell engineering system as desired, and low temperature zone for holding low temperature chamber. The cell engineering system 102 can also include a user interface, which can include a bar code reader, and the ability to receive using inputs by touch pad or other similar device.

As shown in FIG. 1, cassette 100 may further include an additional chamber volume, or satellite volume 165, for increasing the working volume of the cell culture chamber by providing additional volume for media and other working fluids, or can be used to house a cell culture or portion of a cell culture during the activating, transducing, expanding, concentrating, and/or harvesting steps, of said cell culture. Suitably, the satellite volume 165 is oriented substantially perpendicular, and fluidly connected to, the cell culture chamber 101 such that media is exchanged with the culture chamber without disturbing the cell culture. In exemplary embodiments, satellite volume is a bag, and in other embodiments, satellite volume is a non-yielding chamber, such as shown in FIG. 1. In embodiments, the satellite volume is between about 0.50 ml and about 1 L, or about 10 ml and 800 ml, or about 100 ml and about 500 ml, or about 100 ml to about 300 ml, more suitably between about 150 ml and about 200 ml. In embodiments, the satellite volume is further configured to allow media removal without loss of cells of the immune cell culture. That is, the media exchange between the satellite volume and the cell culture chamber is performed in such a manner that the cells are not disturbed and are not removed from the cell culture chamber. In some embodiments, the satellite volume may additionally include a port, inlet, cut-out, lumen, or pass-through, that allows for airflow to be transferred from the outside of the cassette to the internals of the cassette.

As shown in FIG. 1, satellite volume 165 suitably includes a satellite volume lumen 170, which is a passage through the satellite volume 165 that allows for gas to flow through the satellite volume 165, while still maintaining the fluid integrity of satellite volume 165. FIG. 5 shows the orientation of cell culture chamber 101 within cell engineering system 102, with the cell culture chamber oriented substantially horizontally, and satellite volume 165 oriented substantially vertically with a shell 500 of the cell engineering system which is a movable cover surrounding the system. As shown, satellite volume lumen 170 is posited such that air 502 can move through the lumen, into the center of the cell engineering system 102 and cassette 100 to provide increased airflow as well as temperature control and regulation.

Positioning of cell culture chamber 101 in a horizontal position within cell engineering system 102 is carried out by turning the entire cell engineering system 102 by approximately 90 degrees, such that cell culture chamber 101 which was originally oriented vertically (see FIG. 1 with position on cassette 100), to a horizontal position, as shown in FIG. 5. This allows the utilization of a larger volume for cell culture chamber 101, as the face of cassette 100 where the cell culture chamber 101 is positioned is larger than the top section of the cassette.

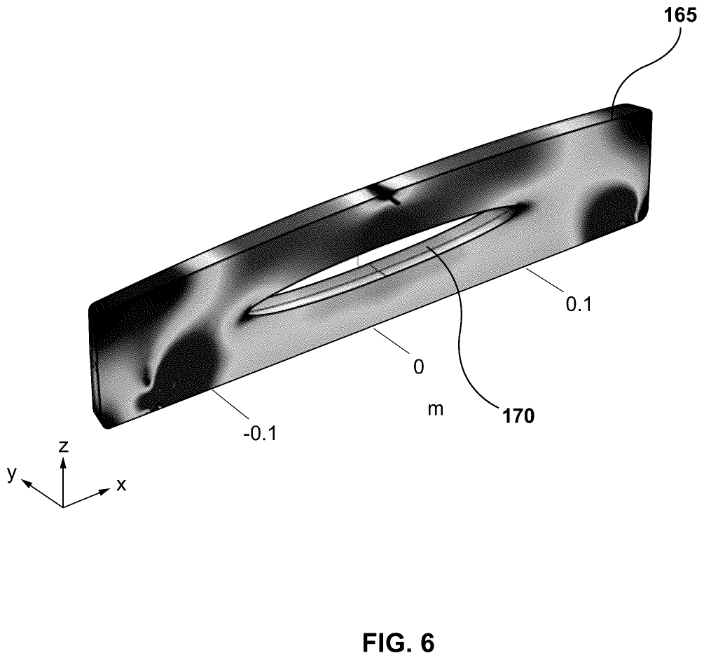

FIG. 6 shows a temperature gradient diagram of satellite volume 165 with satellite volume lumen 170 shown.

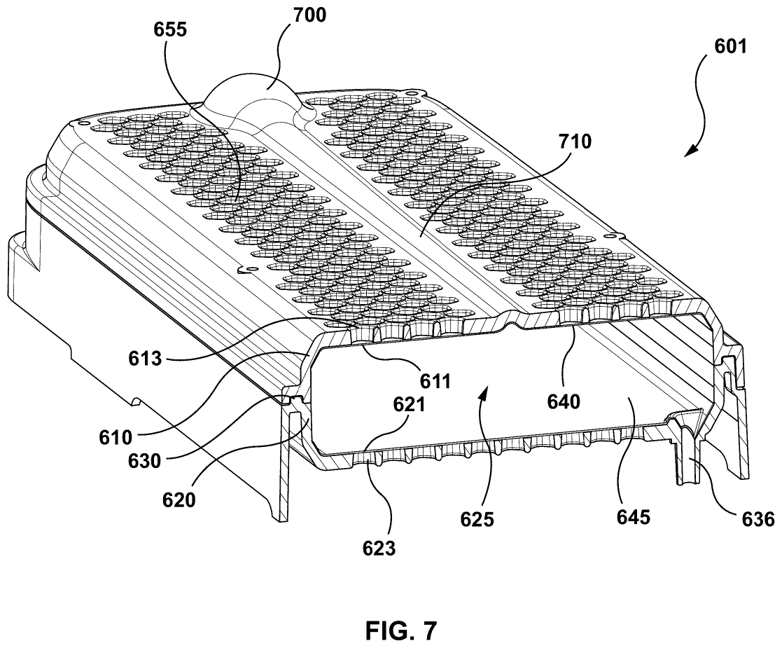

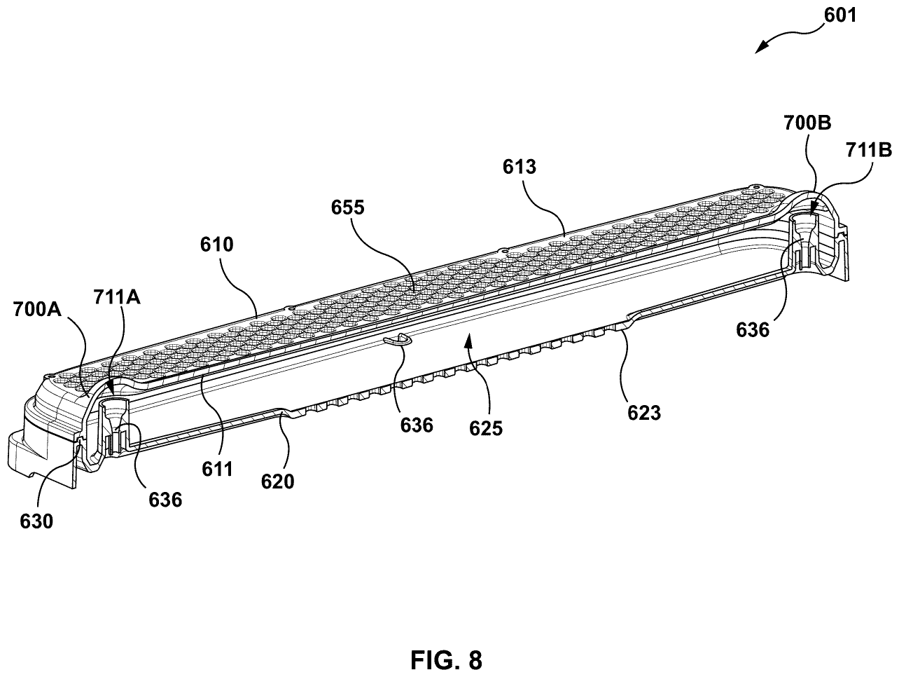

In embodiments, a cell culture chamber 601 for use in an automated cell engineering system includes a first body 610 and a second body 620 configured to couple together, for example, as shown in FIGS. 7-12. The first body 610 forms a first portion 612 of the cell culture chamber 601 and the second body 620 forms a second portion 622 of the cell culture chamber 601. When coupled together, the first body 610 and the second body 620 form an enclosed volume 625, as shown in the cross-sectional views of FIGS. 7 and 8. The cell culture chamber 601 may be formed without any structures disposed within the enclosed volume 625, so as to allow for an increased volume of media disposed within said enclosed volume 625, as shown in FIGS. 7 and 8. A first non-porous, gas-permeable material 640 may be disposed on the first body 610 and a second non-porous, gas-permeable material 645 may be disposed on the second body 620, see FIG. 7. In an embodiment, the first non-porous, gas-permeable material 640 may be formed together with the first body 610, and the second non-porous, gas-permeable material 645 may be formed together with the second body 620, using the forming procedure previously described herein. In embodiments, the first body 610 may not include the first non-porous, gas-permeable material 640, while the second body 620 does include the second non-porous, gas-permeable material 645. Alternatively, the first body 610 does include the first non-porous, gas-permeable material 640, while the second body 620 may not include the second non-porous, gas-permeable material 645. In the embodiments where only one of the first body 610 or the second body 620 includes the non-porous, gas-permeable material 640/645, an advantageous maximization of oxygen levels within the cell culture chamber 601 may be achieved. In another embodiment, the first body 610 may include the first non-porous, gas-permeable material 640, while the second body 620 also includes the second non-porous, gas-permeable material 645.

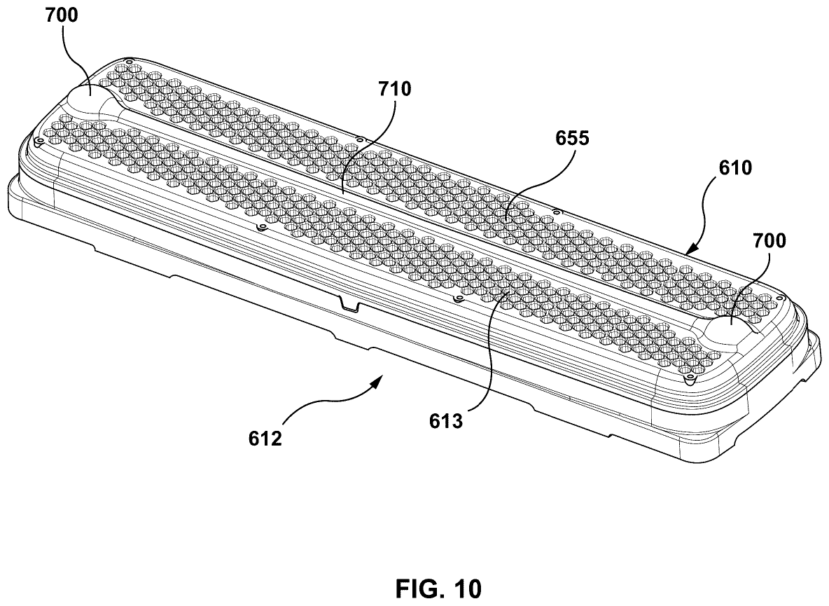

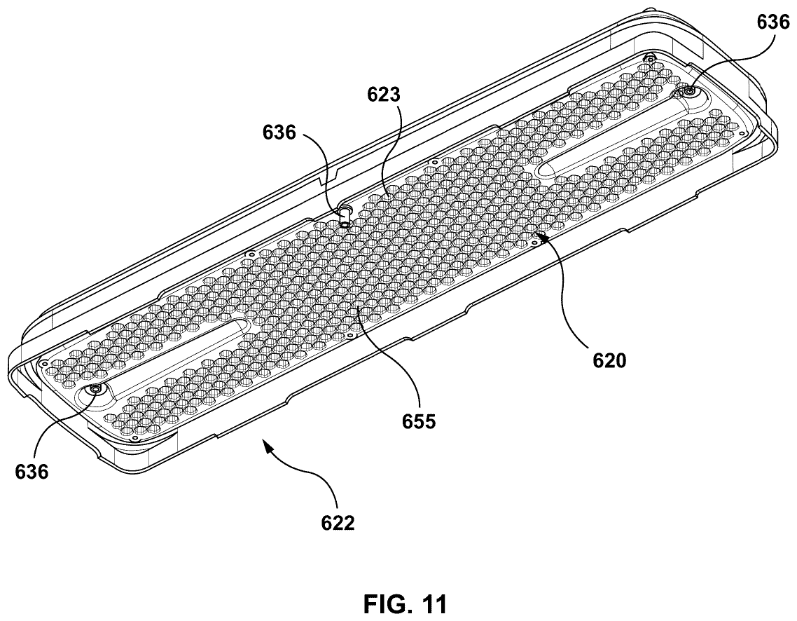

As noted above, the enclosed volume 625 is formed in the area between the first body 610 and the second body 620. The enclosed volume 625 is configurable to contain a cell culture and is configurable to be liquid sealed (does not allow liquid to flow into or out of the enclosed volume, but will allow gas to flow, as described herein), except for where intended through designated valves, ports, and the like. For example, the inlet/outlet ports 636 may connect to other elements of the cassette 100 or cell engineering system 102 so as to provide for the exchange of media or other reagents to and from the enclosed volume 625, and allow for harvest of the cell culture within the enclosed volume 625. As shown in FIGS. 7 and 8, the first body 610 and the second body 620 are coupled together using mating elements, for example at coupling 630, such as snap-fit, form-fit, or other methods known in the art. FIGS. 10 and 11 depict a top perspective view and a bottom perspective view of the cell culture chamber 601 with the first body 610 and the second body 620 coupled together, respectively. Additionally, it is envisioned, that the first body 610 and the second body 620 may be formed as a unitary body, removing the need for the first body 610 and the second body 620 to be coupled to together.

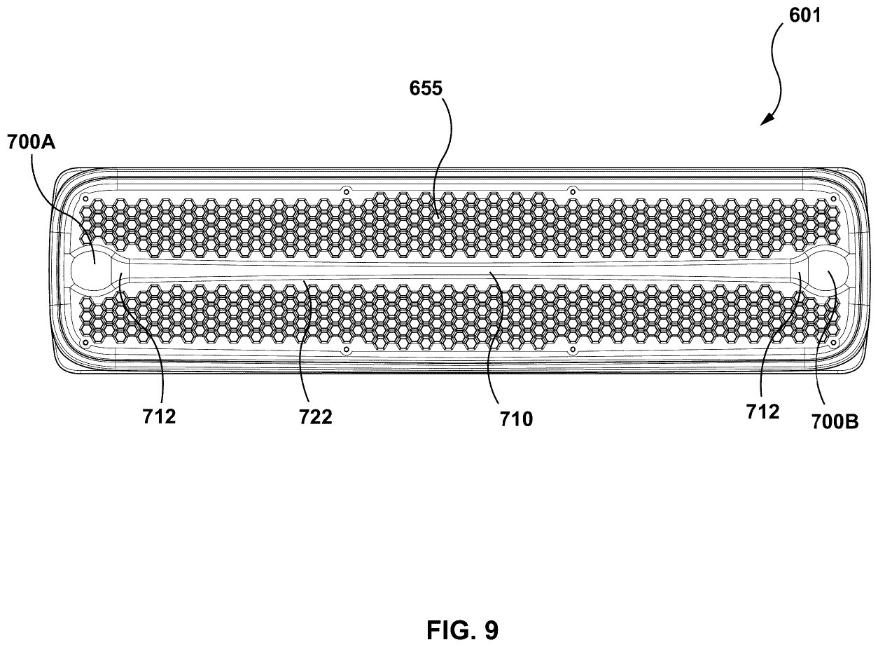

FIG. 9 provides exemplary dimensions for the cell culture chamber 601, in a top-down view. For example, the length of the cell culture chamber 601 (i.e. the length of the first body 610 and/or second body 620) may be approximately 360 mm. In embodiments, the length of the cell culture chamber may be between 200-600 mm. In embodiments, the length of the cell culture chamber may be between 300-400 mm. The width of the cell culture chamber 601 (i.e. the width of the first body 610 and/or second body 620) may be approximately 90 mm. In embodiments, the width of the cell culture chamber 601 may be between 50-150 mm. In embodiments, the width of the cell culture chamber may be between 75-100 mm.

In order to allow gas exchange between the enclosed volume 625 and the outside environment, the first body 610 and/or the second body 620 may include a plurality of openings 655 configured to allow for gas exposure to the external environment via the outside surface of the first non-porous, gas-permeable material 640 and/or the second non-porous, gas-permeable material 645 disposed in the cell culture chamber 601 as shown in FIGS. 7-11. For example, FIGS. 7-10 show the plurality of openings 655 formed within or through, or disposed through the first body 610 in a staggered arrangement, while FIG. 11 shows the plurality of openings 655 formed within or through, or disposed through the second body 620 in a staggered arrangement. The plurality of openings 655 may take a variety of shapes including geometric (e.g. hexagonal, septagonal, octagonal, etc.), symmetrical, honeycomb, round, circular, oblong, oval, square, etc. Additionally, the design of the plurality of openings 655 may be used to provide additional structural support and strength to the first body 610 and the second body 620. For example, the use of a honeycomb structure allows for a high ratio of open space to structure, allowing for a higher volume of gas to be exchanged, while reducing the amount of structure that is required to support the first body 610 and/or the second body 620. In embodiments, the plurality of openings 655 may be formed within or through/arranged on the first body 610 and/or the second body 620 in a sequential arrangement. In embodiments, the first body 610 and/or the second body 620 may instead be formed of a solid thermoplastic without the plurality of openings 655 formed therein. At least about 30% of the first body 610 and/or the second body 620 may be covered in the plurality of openings 655, more suitably at least about 40%, at least about 50%, at least about 60%, at least about 70%, at least about 80%, at least about 90%, or at least about 100% (i.e., the entirety).

An inner surface 611, otherwise known as a media-contacting surface, of the first body 610 and an inner surface 621, or media-contacting surface, of the second body 620 of the cell culture chamber 601 are suitably lined or covered with non-porous gas-permeable material(s) so as to prevent water (i.e. in liquid form) or other liquids (e.g., cell culture media) from evaporating from within and exiting the cell culture chamber 601, while simultaneously allowing for the passage of gasses such as oxygen, nitrogen, carbon dioxide, and the like. As used herein, “a non-porous, gas-permeable material” means any composition, coating, film, or material used for gas-permeable cell culture devices, that allows for gases to pass and enter the cell culture chamber 601, but does not contain pores or holes that allow for passage or leakage of liquids (i.e. cell media). Exemplary non-porous, gas-permeable materials include, but are not limited to, silicone, fluoroethylene polypropylene (FEP), polyolefin, ethyl vinyl olefin (EVO) and ethylene vinyl acetate copolymer. Non-porous, gas-permeable materials as described herein suitably help to deliver one or more gasses, including oxygen, nitrogen, CO₂, etc., to the cells in the cell culture chamber 601. Additionally, the thickness of the non-porous gas-permeable material may be reduced or increased in order to increase or decrease the gas exchange through the material.

As shown in FIG. 7, in embodiments, a first non-porous, gas permeable material 640 is positioned at the inner surface 611 of the first body 610 and a second non-porous, gas permeable material 645 is positioned at the inner surface 621 of the second body 620. During the formation of the cell culture chamber 601, for example, through the use of molding, the first and second non-porous gas-permeable material 640/645 may be simultaneously created at the inner surfaces 611/621 through a dual-shot molding technique. In an embodiment, this molding technique includes an injection molding technique. For example, the first body 610 and second body 620 of the cell culture chamber 601 are formed via a first shot of the dual-shot molding technique. After a brief delay allowing the thermoplastic to harden, a core portion of the mold is retracted in small volumes to create space for a second shot of silicone (or any other suitable gas-permeable material) to form the gas permeable material 640/645 disposed on the first body 610 and second body 620, respectively. The measured thickness of the gas permeable material 640/645 is determinative on the amount of mold retracted from the first shot, and thus can be adjusted in accordance with end user needs. Additionally, the non-porous, gas-permeable materials may be cast onto or into the cell culture chamber 601, to provide the gas-permeable characteristics described herein. Casting of the non-porous, gas-permeable material may be carried out using various methods known in the art. Through the process of casting the non-porous, gas-permeable material onto or into the cell culture chamber 601, the structural strength and integrity of the gas-permeable membrane may be increased, while maintaining gas-permeable characteristics. The non-porous, gas-permeable materials may also be coated, sprayed, painted, layered, or otherwise disposed on the inner surfaces 611/621 of the first 610 and second 620 bodies. The first and second non-porous, gas-permeable layers 640/645 may be constructed of the same or different materials, and, in embodiments, each layer 640/645 may include a plurality of different materials.

In exemplary embodiments, “a portion” of the inner surface, such as 611, comprises the non-porous, gas-permeable material. As used herein, “a portion” refers to at least about 20% of the surface, being made up of the non-porous, gas-permeable material. In embodiments, both media contacting surfaces 611 and 621 comprise the non-porous, gas-permeable material. In embodiments in which less than 100% of the media-contacting surface is made of the non-porous, gas-permeable material, the remainder of the media-contacting surface 611 can include other suitable materials, including various plastics that promote the adhesion and growth of cells (e.g., polypropylene, polystyrol, polystyrene, etc.). In embodiments in which less than 100% of the media-contacting surface is made of the non-porous, gas-permeable material, the remainder of the media-contacting surface 621 can include other suitable materials, including various plastics that provide structural support (e.g., polypropylene, polystyrol, polystyrene, etc.). In embodiments, the inner surface 611 of the first body 610 may lack the non-porous, gas-permeable material, such that none (i.e. 0%) of the inner surface 611 is covered. In embodiments, the inner surface 621 of the second body 620 may lack the non-porous, gas-permeable material, such that none (i.e. 0%) of the inner surface 621 is covered.

In embodiments, at least about 30% of the media-contacting surface is made up of the non-porous, gas-permeable material, more suitably at least about 40%, at least about 50%, at least about 60%, at least about 70%, at least about 80%, at least about 90%, or at least about 100% (i.e., the entirety) of the media-contacting surface comprises the non-porous, gas-permeable material.

In embodiments, the inner surfaces of the first body 610 and the second body 620 may further include a surface coating such as a surface coating that activates a cell; a surface coating that modulates a biological pathway in a cell; a surface coating that enhances growth of a cell; a surface coating that increases transduction efficiency of a cell; a surface coating that improves selection for a certain type of cells; a surface coating that improves adhesion of a cell; a surface coating that inhibits a cell; a surface coating that has controlled solubility. Thus, in embodiments, the non-porous gas-permeable material that suitably makes up a portion or the entirety of the media-contacting surface can further include a surface coating as described herein. In other embodiments, the remainder of the media-contacting surface that does not include a non-porous gas-permeable material can also include a surface coating as described herein. In other embodiments, the entire media-contacting surface does not include a non-porous gas-permeable material, but still includes a surface coating as described herein. The adhesion of a cell can also provide an opportunity for cell selection to promote the growth of a desired population, while not allowing adherence of an undesired population.

In embodiments, the cell culture chamber 601 is a contained, and non-flexible chamber. The use of a non-flexible chamber allows the cells to be maintained in a substantially undisturbed state. As described, cell culture chamber 601 can be oriented so as to allow the cell culture to spread throughout the cell culture chamber 601. In embodiments, the overall thickness of cell culture chamber 601 is on the order of about 0.5 cm to about 5 cm. Suitably, the cell culture chamber may have a volume of between about 0.50 ml and about 1 L, more suitably about 0.5 to about 900 ml, or the cell culture chamber has a volume of about 825 ml. Suitably, the cell culture chamber may have a volume of between about 0.5 ml to about 500 ml, about 10 ml to about 300 ml, more suitably between about 50 ml and about 200 ml, or the cell culture chamber has a volume of about 180 ml. The use of a low chamber height (less than 5 cm, suitably less than 4 cm, less than 3 cm, or less than 2 cm) allows for effective media and gas exchange in close proximity to the cells. Ports are configured to allow mixing via recirculation of the fluid without disturbing the cells. Larger height static vessels can produce concentration gradients, causing the area near the cells to be limited in oxygen and fresh nutrients. Through controlled flow dynamics, media exchanges can be performed without cell disturbance. Media can be removed from the additional chambers (no cells present) without risk of cell loss.

In some embodiments, the cell culture chamber 601 may include inlet/outlet ports 636 or a coupler that may be configured to be connected to an automated cell engineering system. Such components allow for external machinery, such as pumps, tubing, connectors, or other machinery, to interact with and provide media and cells, or additional support to the media and cells within the enclosed volume 625 as well as the components of the automated cell engineering system. In embodiments, the inlet/outlet ports may be configured to receive valves or couplers. Valves can be, for example, trumpet valves—i.e., valves that have two positions (open “0” or closed “1”), or pinch valves that are capable of compressing or pinching flow-paths. The inlet/outlet ports 636 can also include couplers (not shown) that allow for the connection of various syringes, sampling devices, etc.

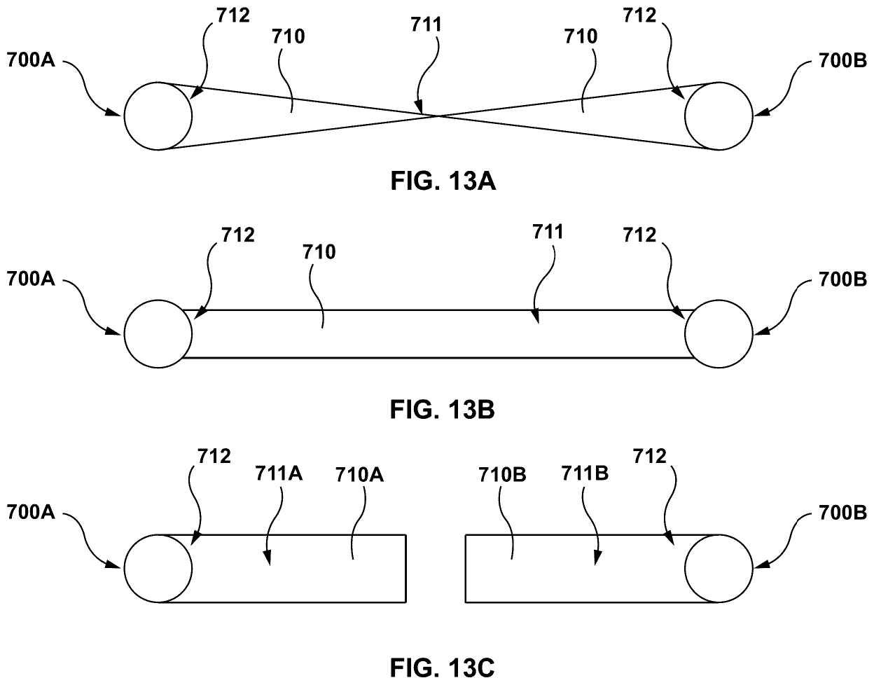

In embodiments, a top surface 613 of the first body 610 includes or is integrally formed with at least one bubble trap 700. The at least one bubble trap 700 is configured to collect any bubbles that may form and rise to the top of the media within the enclosed volume 625 of the cell culture chamber 601. As illustrated in FIG. 9, at least two bubble traps 700A, 700B are disposed on each end (one trap 700A, 700B on each end) of the top surface 613 of the first body 610, with a bubble trap track 710 included with or integrally formed with the first body 610, the bubble trap track 710 further extending between and fluidly connecting each bubble trap 700A, 700B. FIGS. 13A-13B depict various embodiments in which the at least two bubble traps 700A, 700B and bubble trap track 710 may be formed. The bubble trap track 710 may vary in a width 722 along its length, such that the bubble trap track 710 is wider towards its ends 712 (i.e. closer to the bubble traps 700), and narrower towards the middle of its length (i.e. at or near the center of the top surface 613). The bubble trap track 710 may thus taper outwardly from the middle of the bubble trap track 710 to the ends of the bubble trap track 710 adjacent to the at least two bubble traps 700A, 700B. For example, the width 722 of the bubble trap track 710 may be such that the center of the bubble trap track 710 (i.e. near the planar center of the inner surface 611) begins with almost no width/space, and gradually increases in width 722 (tapers in width) along the length of the bubble trap track 710 (i.e. towards the at least one bubble trap 700) by up to 1 mm or more towards the ends 712 of the bubble trap track 710 that meet/connect with the at least one bubble trap 700, thus forming a portion of an additional volume 711, as illustrated in FIG. 13A, for example. In embodiments, the width 722 of the bubble trap track 710 may increase from any value between 0.01 mm to 10 mm in width, starting from the center of the bubble trap track 710 and ending towards the ends 712 of the bubble trap track 710 connected to the at least one bubble trap 700, respectively. In embodiments, the bubble trap track 710 may taper inwardly from the middle of the bubble trap track 710 to the ends of the bubble trap track 710 adjacent to the at least two bubble traps 700A, 700B (not shown). In embodiments, at least two bubble trap tracks 710A, 710B may fluidly connect to each of the at least two bubble traps 700A, 700B, such that each bubble trap 700A, 700B is connected to one bubble trap track 710A, 710B, and each bubble trap track 710A, 710B is disconnected from each other (i.e., there is space between the bubble trap track sections), as illustrated in FIG. 13C, for example. The bubble trap tracks 710A, 710B may taper outwardly starting from the end of the bubble trap tracks furthest from the bubble traps 700A, 700B, as illustrated in FIG. 13A, for example. In embodiments, each bubble trap track 710A, 710B may taper inwardly starting from the end of the bubble trap tracks furthest from the bubble traps 700A, 700B (not shown). In embodiments, the bubble trap tracks 710A, 710B may have a uniform width throughout its length, from the end of the bubble trap track furthest from the bubble trap 700A, 700B, to the end 712 of the bubble trap track 710 closest or fluidly connected to the bubble trap 700A, 700B, as illustrated in FIG. 13B, for example.

In embodiments, the at least one bubble trap 700 is fluidly connected to the bubble trap track 710. In embodiments, the at least two bubble traps 700A, 700B, may each fluidly connect to at least two bubble trap tracks 710A and 710B (FIG. 13C), forming at least two additional volumes 711A, 711B. In embodiments, a plurality of bubble traps 700 may fluidly connect to a plurality of bubble trap tracks 710. In embodiments, a plurality of bubble traps 700 may fluidly connect to the bubble trap track 710. In embodiments, a plurality of bubble traps 700 may fluidly connect to a single bubble trap track 710. For example, the first body 610 may include or be integrally formed with a plurality of bubble traps 700, and a bubble trap track 710 fluidly connected to each of the plurality of bubble traps 700. In embodiments, the plurality of bubble traps 700 may fluidly connect to respective bubble trap tracks 710, such that the bubble trap tracks 710 are disconnected from each other.

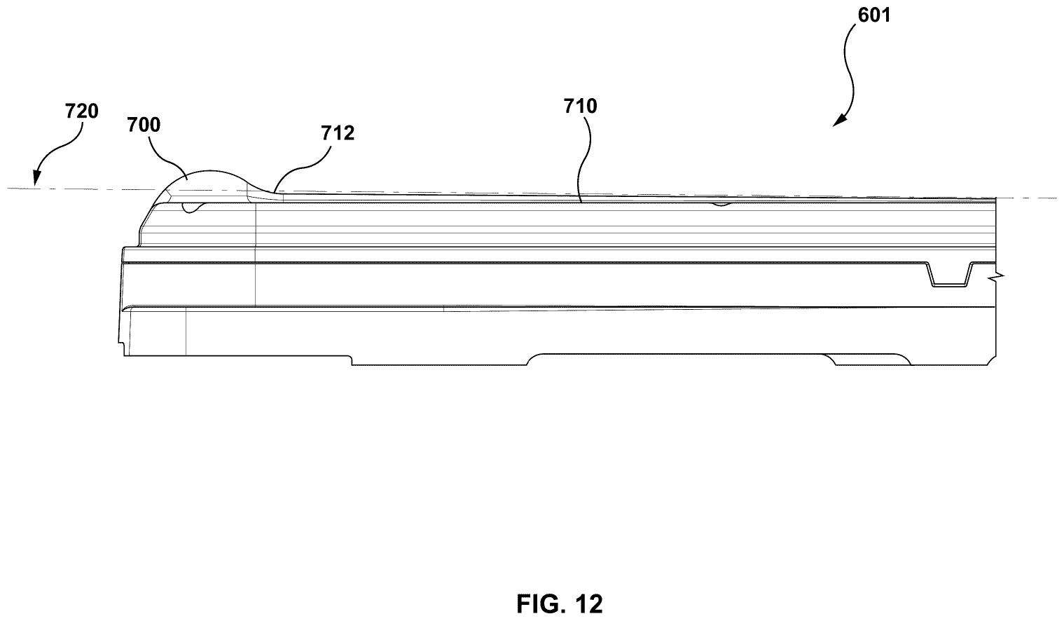

The bubble trap track 710/710A/710B may also be structured with a gradually increasing height. For example, FIGS. 8 and 12 show the bubble trap track 710 formed with a slight gradient 720 on the inner surface 611 of the first body 610, so as to guide bubbles collected closer to the center of the bubble trap track 710 (i.e. near the planar center of the inner surface 611 of the first body 610) outwards towards the at least one bubble trap 700. In FIG. 12, it is exemplified that the gradient 720 of the bubble trap track 710 may be such that the center of the bubble trap track 710 (i.e. near the planar center of the inner surface 611) begins with almost no height/space, and gradually increases in height along the length of the bubble trap track 710 (i.e. tapers in height towards the at least one bubble trap 700) by up to 1 mm or more towards the ends 712 of the bubble trap track 710 that meet/connect with the at least one bubble trap 700, thus forming a portion of the additional volume 711. The bubble trap track 710 may thus taper away from the middle of the bubble trap track 710 to the ends of the bubble trap track 710 adjacent to the at least two bubble traps 700A, 700B. In embodiments, the gradient 720 of the bubble trap track 710 may increase from any value between 0.01 mm to 10 mm in height on the inner surface 611, starting from the center of the bubble trap track 710 and ending towards the ends 712 of the bubble trap track 710 connected to the at least one bubble trap 700, respectively. The at least one bubble trap 700 and bubble trap track 710 may further form additional volume 711 within the enclosed volume 625 for collecting/holding the bubbles described herein. This additional volume 711 is structured only for the collection of bubbles formed within the media disposed within the enclosed volume 625, such that the fluid height of media within the enclosed volume 625 remains below 20 mm (i.e., media does not enter the bubble trap track 710 nor the at least one bubble trap 700). The curved, long profile (i.e. the gradient 720) of the bubble trap track 710 helps to guide bubbles along the length of the bubble trap track 710 into the at least one bubble trap 700 located at the end of the bubble trap track 710.

In embodiments, the at least one bubble trap 700 described herein may be included or integrally formed with a bottom surface 623 of the second body 620 (not shown). A plurality of bubble traps 700 may be disposed on each end of the bottom surface 623 of the second body, with the bubble trap track 710 having the gradient 720 running between and connecting to each bubble trap 700. The bubble trap track 710 may vary in width along its length and in its gradient 720, as previously described herein. This configuration would allow for the at least one bubble trap 700 and bubble trap track 710 to collect bubbles formed within the media in the event the cell culture chamber is flipped 601 (i.e. axially rotated), such that the second body 620 is facing upwards. The bubble traps 700/700A/700B and bubble trap tracks 710/710A/710B may be included or integrally formed with the second body 620 in a similar manner as previously described above with respect to the first body 610.

While various embodiments according to the present invention have been described above, it should be understood that they have been presented by way of illustration and example only, and not limitation. It will be apparent to person skilled in the relevant art that various changes in form and detail can be made therein without departing from the spirit and scope of the invention. Thus, the breadth and scope of the present invention should not be limited by any one of each embodiment discussed herein, and of each reference cited herein, can be used in combination with the features of any other embodiment. All patents and publication discussed herein are incorporated by reference herein in their entirety.

Embodiments of the present disclosure include the following examples.

Example 1. A cell culture chamber for use in an automated cell engineering system, the cell culture chamber comprising: a first body forming a first portion of the cell culture chamber; a second body forming a second portion of the cell culture chamber, the first body and the second body configured to couple together, thereby forming an enclosed volume; wherein a first non-porous, gas-permeable material is disposed on the first body; and wherein the first non-porous, gas-permeable material and the first body are formed together.

Example 2. The cell culture chamber of example 1, further including a second non-porous, gas-permeable material disposed on the second body; and wherein the second non-porous, gas-permeable material and the second body are formed together.

Example 3. The cell culture chamber of example 1, further including at least one bubble trap integrally formed with the cell culture chamber.

Example 4. The cell culture chamber of example 1, further comprising one or more structural islands.

Example 5. The cell culture chamber of example 2, wherein the first body and the second body couple together using mating elements.

Example 6. The cell culture chamber of example 2, wherein the first body includes a plurality of openings configured to allow gas exposure to an external environment via an outside surface of the first non-porous, gas-permeable material and/or the second non-porous, gas-permeable material.

Example 7. The cell culture chamber of example 6, wherein the plurality of openings comprise a geometric, hexagonal, septagonal, octagonal, symmetrical, honeycomb, round, circular, oblong, oval, or square shape.

Example 8. The cell culture chamber of example 6, wherein the first non-porous, gas-permeable material is positioned on an inner surface of the first body, the first non-porous, gas-permeable material being configured to cover the plurality of openings of the first body.

Example 9. The cell culture chamber of example 2, wherein a structural section of the second body includes a plurality of openings configured to allow gas exposure to an external environment via an outside surface of the first non-porous, gas-permeable material and/or the second non-porous, gas-permeable material.

Example 10. The cell culture chamber of example 9, wherein the plurality of openings comprise a geometric, hexagonal, septagonal, octagonal, symmetrical, honeycomb, round, circular, oblong, oval, or square shape.

Example 11. The cell culture chamber of example 10, wherein the second non-porous, gas-permeable material is positioned on an inner surface of the first body, the second non-porous, gas-permeable material being configured to cover the plurality of openings of the second body.

Example 12. The cell culture chamber of example 3, wherein the at least one bubble trap is integrally formed with the first body of the cell culture chamber, and further including a bubble trap track integrally formed with the first body and fluidly connected to the at least one bubble trap.

Example 13. The cell culture chamber of example 12, wherein at least two bubble traps are integrally formed with the first body of the cell culture chamber, and the bubble trap tracks are fluidly connected to the at least two bubble traps.

Example 14. The cell culture chamber of example 13, wherein the bubble trap track tapers in width.

Example 15. The cell culture chamber of example 13, wherein the bubble trap track tapers in height.

Example 16. The cell culture chamber of example 12, wherein at least two bubble traps are integrally formed with the first body of the cell culture chamber, and at least two corresponding bubble trap tracks are fluidly connected to each of the at least two bubble traps such that the at least two corresponding bubble trap tracks are disconnected from each other.

Example 17. An automated cell engineering system, comprising: a cassette including: a cell culture chamber having at least one bubble trap disposed thereon, and further including: a first body forming a first portion of the cell culture chamber; a second body forming a second portion of the cell culture chamber, the first body and the second body configured to couple together, thereby forming an enclosed volume; wherein a first non-porous, gas-permeable material is disposed on the first body and/or a second non-porous, gas-permeable material is disposed on the second body; and wherein the first non-porous, gas-permeable material and the first body are formed together and the second non-porous, gas-permeable material and the second body are formed together.

Example 18. The automated cell engineering system of example 17, the cell culture chamber is in a horizontal orientation.

Example 19. The automated cell engineering system of example 17, wherein the cell culture chamber includes a plurality of bubble traps disposed thereon.

Example 20. The automated cell engineering system of example 17, wherein the first body includes a plurality of openings, and the second body includes a plurality of openings.

Example 21. The automated cell engineering system of example 20, wherein the first non-porous, gas-permeable material is positioned on an inner surface of the first body, the first non-porous, gas-permeable material being configured to cover the plurality of openings of the first body.

Example 22. The automated cell engineering system of example 20, wherein the second non-porous, gas-permeable material is positioned on an inner surface of the second body, the second non-porous, gas-permeable material being configured to cover the plurality of openings of the second body.

Example 23. An automated cell engineering system comprising: a cell culture chamber including: a first body forming a first portion of the cell culture chamber and having a plurality of bubble traps disposed thereon, and a bubble trap track disposed between and connected to the plurality of bubble traps; a second body forming a second portion of the cell culture chamber, the first body and the second body configured to couple together, thereby forming an enclosed volume; wherein a first non-porous, gas-permeable material is disposed on the first body and a second non-porous, gas-permeable material is disposed on the second body; and wherein the first non-porous, gas-permeable material and the first body are formed together and the second non-porous, gas-permeable material and the second body are formed together.

Example 24. The automated cell engineering system of example 23, wherein the cell culture chamber is in a horizontal orientation.

Example 25. The automated cell engineering system of example 24, wherein the bubble trap track tapers in height and/or tapers in width.

Example 26. The automated cell engineering system of example 25, wherein each of the plurality of bubble traps are formed at opposite ends of the first body.

Example 27. The automated cell engineering system of example 26, wherein the first non-porous, gas-permeable material is positioned on an inner surface of the first body, the first non-porous, gas-permeable material being configured to cover a plurality of openings of the first body.

Example 28. The automated cell engineering system of example 27, wherein the second non-porous, gas-permeable material is positioned on an inner surface of the second body, the second non-porous, gas-permeable material being configured to cover the plurality of openings of the second body.

Example 29. A cell culture chamber for use in an automated cell engineering system, the cell culture chamber comprising: at least one body forming an enclosed volume; and a non-porous, gas-permeable material disposed on the at least one body such that the non-porous, gas-permeable material and the at least one body are formed together; wherein no structures are disposed within the enclosed volume.

1. A cell culture chamber for use in an automated cell engineering system, the cell culture chamber comprising:
a first body forming a first portion of the cell culture chamber;
a second body forming a second portion of the cell culture chamber, the first body and the second body configured to couple together, thereby forming an enclosed volume;
wherein a first non-porous, gas-permeable material is disposed on the first body; and
wherein the first non-porous, gas-permeable material and the first body are formed together.

2. The cell culture chamber of claim 1, further including a second non-porous, gas-permeable material disposed on the second body; and
wherein the second non-porous, gas-permeable material and the second body are formed together.

3. The cell culture chamber of claim 1, further including at least one bubble trap integrally formed with the cell culture chamber.

4. The cell culture chamber of claim 1, further comprising one or more structural islands.

5. The cell culture chamber of claim 2, wherein the first body and the second body couple together using mating elements.

6. The cell culture chamber of claim 2, wherein the first body includes a plurality of openings configured to allow gas exposure to an external environment via an outside surface of the first non-porous, gas-permeable material and/or the second non-porous, gas-permeable material.

7. The cell culture chamber of claim 6, wherein the plurality of openings comprise a geometric, hexagonal, septagonal, octagonal, symmetrical, honeycomb, round, circular, oblong, oval, or square shape.

8. The cell culture chamber of claim 6, wherein the first non-porous, gas-permeable material is positioned on an inner surface of the first body, the first non-porous, gas-permeable material being configured to cover the plurality of openings of the first body.

9. The cell culture chamber of claim 2, wherein a structural section of the second body includes a plurality of openings configured to allow gas exposure to an external environment via an outside surface of the first non-porous, gas-permeable material and/or the second non-porous, gas-permeable material.

10. The cell culture chamber of claim 9, wherein the plurality of openings comprise a geometric, hexagonal, septagonal, octagonal, symmetrical, honeycomb, round, circular, oblong, oval, or square shape.

11. The cell culture chamber of claim 10, wherein the second non-porous, gas-permeable material is positioned on an inner surface of the first body, the second non-porous, gas-permeable material being configured to cover the plurality of openings of the second body.

12. The cell culture chamber of claim 3, wherein the at least one bubble trap is integrally formed with the first body of the cell culture chamber, and further including a bubble trap track integrally formed with the first body and fluidly connected to the at least one bubble trap.

13. The cell culture chamber of claim 12, wherein at least two bubble traps are integrally formed with the first body of the cell culture chamber, and the bubble trap tracks are fluidly connected to the at least two bubble traps.

14. The cell culture chamber of claim 13, wherein the bubble trap track tapers in width.

15. The cell culture chamber of claim 13, wherein the bubble trap track tapers in height.

16. The cell culture chamber of claim 12, wherein at least two bubble traps are integrally formed with the first body of the cell culture chamber, and at least two corresponding bubble trap tracks are fluidly connected to each of the at least two bubble traps such that the at least two corresponding bubble trap tracks are disconnected from each other.

17. An automated cell engineering system, comprising:
a cassette including:
a cell culture chamber having at least one bubble trap disposed thereon, and further including:
a first body forming a first portion of the cell culture chamber;
a second body forming a second portion of the cell culture chamber, the first body and the second body configured to couple together, thereby forming an enclosed volume;
wherein a first non-porous, gas-permeable material is disposed on the first body and/or a second non-porous, gas-permeable material is disposed on the second body; and
wherein the first non-porous, gas-permeable material and the first body are formed together and the second non-porous, gas-permeable material and the second body are formed together.

18. The automated cell engineering system of claim 17, the cell culture chamber is in a horizontal orientation.

19. The automated cell engineering system of claim 17, wherein the cell culture chamber includes a plurality of bubble traps disposed thereon.

20. The automated cell engineering system of claim 17, wherein the first body includes a plurality of openings, and the second body includes a plurality of openings.

21. The automated cell engineering system of claim 20, wherein the first non-porous, gas-permeable material is positioned on an inner surface of the first body, the first non-porous, gas-permeable material being configured to cover the plurality of openings of the first body.

22. The automated cell engineering system of claim 20, wherein the second non-porous, gas-permeable material is positioned on an inner surface of the second body, the second non-porous, gas-permeable material being configured to cover the plurality of openings of the second body.

23. An automated cell engineering system comprising:
a cell culture chamber including:
a first body forming a first portion of the cell culture chamber and having a plurality of bubble traps disposed thereon, and a bubble trap track disposed between and connected to the plurality of bubble traps;
a second body forming a second portion of the cell culture chamber, the first body and the second body configured to couple together, thereby forming an enclosed volume;
wherein a first non-porous, gas-permeable material is disposed on the first body and a second non-porous, gas-permeable material is disposed on the second body; and
wherein the first non-porous, gas-permeable material and the first body are formed together and the second non-porous, gas-permeable material and the second body are formed together.

24. The automated cell engineering system of claim 23, wherein the cell culture chamber is in a horizontal orientation.

25. The automated cell engineering system of claim 24, wherein the bubble trap track tapers in height and/or tapers in width.

26. The automated cell engineering system of claim 25, wherein each of the plurality of bubble traps are formed at opposite ends of the first body.

27. The automated cell engineering system of claim 26, wherein the first non-porous, gas-permeable material is positioned on an inner surface of the first body, the first non-porous, gas-permeable material being configured to cover a plurality of openings of the first body.

28. The automated cell engineering system of claim 27, wherein the second non-porous, gas-permeable material is positioned on an inner surface of the second body, the second non-porous, gas-permeable material being configured to cover the plurality of openings of the second body.

29. A cell culture chamber for use in an automated cell engineering system, the cell culture chamber comprising:
at least one body forming an enclosed volume; and
a non-porous, gas-permeable material disposed on the at least one body such that the non-porous, gas-permeable material and the at least one body are formed together;
wherein no structures are disposed within the enclosed volume.
